(19) United States
(12) Patent Application Publication
(10) Pub. No.: US 2015/0118292 A1
(43) Pub. Date: Apr. 30, 2015

(54) COMPOSITIONS AND METHODS FOR TREATMENT OF HAIR LOSS

(71) Applicant: Daniel Khesin, Boca raton, FL (US)

(72) Inventor: Daniel Khesin, Boca raton, FL (US)

(21) Appl. No.: 14/062,108

(22) Filed: Oct. 24, 2013

(51) Int. Cl. A61K 8/63; A61Q 7/00; A61K 8/49

(52) U.S. Cl. CPC A61K 8/63; A61K 8/4953; A61Q 7/00
USPC 424/451; 514/170

(57) ABSTRACT

The present invention is directed to compositions and methods suitable for the treatment of alopecia by topical application include minoxidil sulfate, finasteride, and 17α-estradiol. The combination is characterized by low alcohol content to minimize skin irritation and may be enhanced with the addition of vitamins, minerals, and peptides. The topical use of this combination leads to a lower overall risk of side effects than orally administered drugs.

### FIELD OF THE INVENTION

The invention is directed to compositions, methods and formulations for treatment of hair loss. More particularly, the present invention relates to compositions and methods including minoxidil sulfate, finasteride, and 17α-estradiol for treatment of hair loss by topical administration.

### BACKGROUND OF THE INVENTION

Hair loss is a common skin disorder that affects hair follicles and is characterized by thinning, typically starting at the temples or the crown in men and parietal region in women; continued thinning without treatment leads to atrophy and total loss of hair follicles, which leads to baldness. While the condition is not life threatening and does not endanger health, it leads to social embarrassment and psychological consequences for many sufferers.

A variety of hair loss treatment methods have been developed, including topical minoxidil, oral finasteride, laser therapy, corticosteroid injections, oral contraceptives, various anti-androgens, and surgical procedures such as hair transplantation. The uses of existing therapies, however, have certain disadvantages.

Oral finasteride, an effective treatment for many patients, has had a significant number of reported side effects including decreased libido, erectile dysfunction, ejaculatory dysfunction, and myopathy. The Food and Drug Administration (FDA) has additionally issued a statement that the use of finasteride and duatsteride both increase the risk of high grade prostate cancer. Whether or not these side effects are true or exaggerated there is a fear among many patients to initiate or continue treatment.

Another popular treatment is topical minoxidil. Topical minoxidil is a formulation that contains a high concentration of solvents such as alcohol and propylene glycol both of which lead to skin irritation which reduces patient compliance and reduces the efficacy of the treatment itself (alopecia is often characterized by chronic inflammation). Both topical minoxidil and finasteride are also a single treatment modality; finasteride inhibits the conversion of testosterone to Dihydrotestosterone (DHT) and minoxidil behaves as an ion-channel opener. However, latest research shows that alopecia is caused and triggered by a significant number of variables including exposure to stress, chronic inflammation, perifolicular fibrosis, over-expression or suppression of certain scalp enzymes, auto immune reactions, certain vitamin and mineral deficiencies, sensitivity to DHT, and many more. In addition, there are many more mechanisms involved in alopecia which have not yet been identified. Therefore, it is apparent that while topical minoxidil and oral finasteride have proven efficacy, there address only a single variable and many patients will generally have only partial improvement and not complete improvement of hair loss.

The risks associated with hair transplantation are well known and its usefulness is limited by the number of hair grafts that can be transplanted to the affected area. Patients who have little hair loss and have a successful transplant still require ongoing topical or oral therapy to prevent the surrounding hair (the non-transplanted hairs) from falling out. These patients are also subjected to the disadvantages of topical minoxidil and oral finasteride.

For these reasons, it would be desirable to provide improved compositions and methods for the treatment of alopecia. In particular, it would be desirable to provide products which combine multiple mechanisms of action, with none or fewer of the disadvantages described above.

### BRIEF SUMMARY OF THE INVENTION

In a first embodiment, the invention is directed to a pharmaceutical composition for topical application useful for preventing hair loss, stimulating hair growth, treating alopecia, and thickening hair, including (i) 5-alpha-reductase suppressant (ii) an ion channel opener and (iii) 17-alpha estradiol, wherein the (i) 5-alpha-reductase suppressant (ii) an ion channel opener and (iii) 17-alpha estradiol are combined in a pharmaceutically acceptable fluid carrier.

In another embodiment, the invention is directed to a method to prepare a pharmaceutical composition for topical application useful for preventing hair loss, stimulating hair growth, treating alopecia, and thickening hair including the steps of a. providing a pharmaceutical carrier, b. mixing (i) 5-alpha-reductase suppressant (ii) an ion channel opener and (iii) 17-alpha estradiol in the pharmaceutical carrier and c. depositing the pharmaceutical carrier including the (i) 5-alpha-reductase suppressant (ii) an ion channel opener and (iii) 17-alpha estradiol in a disperserment storage container.

In another embodiment, the invention is directed to a method for topical application useful for preventing hair loss, stimulating hair growth, treating alopecia, and thickening hair including the steps of a. providing a pharmaceutical carrier, b. mixing (i) 5-alpha-reductase suppressant (ii) an ion channel opener and (iii) 17-alpha estradiol in the pharmaceutical carrier c. depositing the pharmaceutical carrier having the (i) 5-alpha-reductase suppressant (ii) an ion channel opener and (iii) 17-alpha estradiol in a disperserment storage container, and d. applying the pharmaceutical carrier including the (i) 5-alpha-reductase suppressant (ii) an ion channel opener and (iii) 17-alpha estradiol in a disperserment storage container to a required area for treatment as directed.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The embodiments discussed herein are not intended to be exhaustive or to limit the invention to the precise form disclosed. These embodiments are chosen and described to best explain the principle of the invention and its application and practical use and to enable others skilled in the art to best utilize the invention.

In a first embodiment, the invention is directed to a pharmaceutical composition for topical application useful for preventing hair loss, stimulating hair growth, treating alopecia, and thickening hair, including (i) 5-alpha-reductase suppressant (ii) an ion channel opener and (iii) 17-alpha estradiol, wherein the (i) 5-alpha-reductase suppressant (ii) an ion channel opener and (iii) 17-alpha estradiol are combined in a pharmaceutically acceptable fluid carrier.

The present invention provides novel hair loss treatment compositions both including minoxidil sulfate (an ion channel opener), finasteride (5α-reductase inhibitor), and 17α-estradiol (secondary 5α-reductase inhibitor). 17α-estradiol differs from finasteride in several respects. It is a stereoisomer of the female hormone 17β-estradiol that inhibits the conversion of testosterone to DHT by suppressing 5α-reductase activity. In addition, by inhibiting 17β-dehydrogenase, it impedes the conversion process of androstenedione to testosterone, resulting in a reduction in the syntheses of testosterone and DHT. It also accelerates the conversion of testosterone to estradiol by stimulating aromatase, decreasing the level of testosterone and leading to a reduction in DHT. The result is broad activity against the main triggers of hair loss. These agents are combined in a pharmaceutically acceptable fluid carrier which has been found to provide effective topical treatment of hair loss.

The minoxidil sulfate will be present in the carrier at a concentration from 5% by weight to 15% by weight, finasteride will be present at a concentration from 0.01% to 0.5% by weight, and 17α-estradiol will be present at a concentration of 0.01% to 0.1% by weight. By maintaining the compositions at a pH below 7, the tendency of minoxidil sulfate to oxidize and degrade finasteride is largely overcome and the product remains stable during storage at room temperature for extended periods, typically several months or longer. Additionally, the compositions of the present invention have been found to remain substantially odor free even after storage at room temperature for extended period.

In a particular aspect of the present invention, the use of minoxidil sulfate has significant advantages over minoxidil, which is widely available in many over the counter products. The main distinction between these two compounds is that minoxidil is an oil soluble molecule while minoxidil sulfate is water soluble. The former requires the uses of heavy solvents such as propylene glycol and alcohol which often leads to irritation of the patients scalp. The latter, minoxidil sulfate allows for an almost entirely water mixture wherein the sulfate ion separates during mixing leaving behind pure minoxidil in a water suspension. Not only does this increase patient comfort and reduce irritation, but also leads to a more successful treatment as irritation of the scalp is itself a cause of Alopecia.

Preparation of the topical compositions by combining these three compounds has a number of advantages. In addition to delivering this composition in a vehicle that is gentler to skin, the three compounds act broadly to deliver multiple mechanisms of action to support optimal functioning of the hair follicle. Due to the fact that the treatment is administered topically, there is a much lower chance of side effects than from oral finasteride and provides an important additional tool for physicians. According to the present invention, topical compositions for the treatment of hair loss include minoxidil sulfate, finasteride and 17α-estradiol present in a fluid carrier or vehicle which is formulated to enhance stability, efficacy, and aesthetic acceptability of the compositions.

The minoxidil sulfate constituent will be pharmaceutical grade. It may be in the form of a finely divided powder, typically having a mean particle size of 40 μm, or lower, or in the form of a hydrous granular material which will have its particle size reduced accordingly during processing according to this invention. Preparation of suitable minoxidil sulfate constituents is well described in the medical and patent.

The finasteride constituent will be pharmaceutical grade. It may be in the form of a finely divided powder, typically having a mean particle size of 35 μm, or lower, or in the form of a hydrous granular material which will have its particle size reduced accordingly during processing according to this invention. Preparation of suitable finasteride constituents is well described in the medical and

17α-estradiol constituent will be pharmaceutical grade. It may be in the form of a finely divided powder, typically having a mean particle size of 25 μm, or lower, or in the form of a hydrous granular material which will have its particle size reduced accordingly during processing according to this invention. Preparation of suitable 17α-estradiol constituents is well described in the medical and scientific journals.

The 17α-estradiol, finasteride, and minoxidil sulfate constituents will be combined in a suitable fluid vehicle or carrier, typically an aqueous carrier, but can also be supplied in the form of a gel, cream, lotion, and mixtures thereof. Exemplary polymers include polyacrylamide (CAS Registry No. 9003-05-8) that is available in a mixture containing polyacrylamide & C13-14 isoparaffin & laureth-7 which is commercially available under the tradename Sepigel, Puteaux Cedex, France. Suitable gelling agents include cellulosic polymers, such as gum arabic, gum tragacanth, locust bean gum, guar gum, xanthan.gum, cellulose gum, methylcellulose, hydroxyethylcellulose, hydroxypropylcellulose, and hydroxypropylmethylcellulose. The goal of a carrier is to deliver the active molecules to the base of the hair follicle (dermal papilla). Each carrier is evaluated for absorption into skin using various methods including the Franz diffusion method.

Other ingredients which may optionally be provided in the topical compositions include vitamins, minerals, botanical extracts, humectants, such as propylene glycol; solvents, such as alcohol; and anti-microbial preservatives, such as methylparaben, propylparaben, phenoxyethanol. The topical compositions will also include an organic or inorganic acids, such as lactic acid, which is used to adjust the pH of the initial components and the final product.

In another embodiment, the invention is directed to a method to prepare a pharmaceutical composition for topical application useful for preventing hair loss, stimulating hair growth, treating alopecia, and thickening hair including the steps of a. providing a pharmaceutical carrier, b. mixing (i) 5-alpha-reductase suppressant (ii) an ion channel opener and (iii) 17-alpha estradiol in the pharmaceutical carrier and c. depositing the pharmaceutical carrier having the (i) 5-alpha-reductase suppressant (ii) an ion channel opener and (iii) 17-alpha estradiol in a disperserment storage container.

The components could be added to any aqueous solution, gel, or cream and mixed together. The storage container can be a spray, dropper, or lotion pump. The alcohol content of the pharmaceutical carrier is 1.0 to 30.0 percent by weight.

In another embodiment, the invention is directed to a method for topical application useful for preventing hair loss, stimulating hair growth, treating alopecia, and thickening hair having the steps of a. providing a pharmaceutical carrier, b. mixing (i) 5-alpha-reductase suppressant (ii) an ion channel opener and (iii) 17-alpha estradiol in the pharmaceutical carrier; c. depositing the pharmaceutical carrier comprising the (i) 5-alpha-reductase suppressant (ii) an ion channel opener and (iii) 17-alpha estradiol in a disperserment storage container, and d. applying the pharmaceutical carrier comprising the (i) 5-alpha-reductase suppressant (ii) an ion channel opener and (iii) 17-alpha estradiol in a disperserment storage container to a required area for treatment as needed.

Treatment should be applied twice per day, morning and night; research illustrates, the first results appear in 90 days. Treatment must be continued indefinitely to maintain results if the patient is affected by male pattern alopecia. If hair loss is caused by other reasons then the treatment should only be used as needed.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A pharmaceutical composition for topical application useful for preventing hair loss, stimulating hair growth, treating alopecia, and thickening hair, comprising (i) 5-alpha-reductase suppressant (ii) an ion channel opener and (iii) 17-alpha estradiol,
wherein the (i) 5-alpha-reductase suppressant (ii) an ion channel opener and (iii) 17-alpha estradiol are combined in a pharmaceutically acceptable fluid carrier.

2. The pharmaceutical composition of claim 1, wherein the compositions is maintained at a pH below 7.

3. The pharmaceutical composition of claim 2, wherein the (i) 5-alpha-reductase suppressant (ii) an ion channel opener and (iii) 17-alpha estradiol each have active components, wherein the active components act simultaneously to prevent hair loss, stimulate hair growth, treat alopecia, and thicken hair.

4. The pharmaceutical composition of claim 3, wherein pharmaceutical composition remains stable during storage at room temperature for at least several months.

5. The pharmaceutical composition of claim 4, the compositions remain substantially odor free during storage at room temperature for at least several months.

6. The pharmaceutical composition of claim 5, wherein the ion channel opener will be present in the carrier at a concentration from 5% by weight to 15% by weight.

7. The pharmaceutical composition of claim 6, wherein the 5-alpha-reductase suppressant will be present at a concentration from 0.01% to 0.5% by weight.

8. The pharmaceutical composition of claim 7, wherein the 17α-estradiol will be present at a concentration of 0.01% to 0.1% by weight.

9. The pharmaceutical composition of claim 8, wherein the ion channel opener is minoxidil sulfate.

10. The pharmaceutical composition of claim 9, wherein the 5-alpha-reductase suppressant is finesteride.

11. The pharmaceutical composition of claim 10, wherein the fluid carrier is an encapsulation system.

12. The pharmaceutical composition of claim 10, wherein the fluid carrier comprises additional components selected from the group comprising vitamins, minerals, or peptides.

13. The pharmaceutical composition of claim 10, wherein the fluid carrier has an alcohol content of 1.0 to 30.0 percent by weight.

14. The pharmaceutical composition of claim 1 or 10, wherein the 17α-estradiol is a stereoisomer of female hormone 17β-estradiol that inhibits the conversion of testosterone to DHT by suppressing 5α-reductase activity and impedes the conversion process of androstenedione to testosterone, resulting in a reduction in the syntheses of testosterone and DHT and accelerates the conversion of testosterone to estradiol by stimulating aromatase, decreasing the level of testosterone and leading to a reduction in DHT.

15. A method to prepare a pharmaceutical composition for topical application useful for preventing hair loss, stimulating hair growth, treating alopecia, and thickening hair comprising the steps of:
a. providing a pharmaceutical carrier;
b. mixing (i) 5-alpha-reductase suppressant (ii) an ion channel opener and (iii) 17-alpha estradiol in the pharmaceutical carrier; and
c. depositing the pharmaceutical carrier comprising the (i) 5-alpha-reductase suppressant (ii) an ion channel opener and (iii) 17-alpha estradiol in a disperserment storage container.

16. A method to for topical application useful for preventing hair loss, stimulating hair growth, treating alopecia, and thickening hair comprising the steps of:
a. providing a pharmaceutical carrier;
b. mixing (i) 5-alpha-reductase suppressant (ii) an ion channel opener and (iii) 17-alpha estradiol in the pharmaceutical carrier; and
c. depositing the pharmaceutical carrier comprising the (i) 5-alpha-reductase suppressant (ii) an ion channel opener and (iii) 17-alpha estradiol in a disperserment storage container; and
d. applying the pharmaceutical carrier comprising the (i) 5-alpha-reductase suppressant (ii) an ion channel opener and (iii) 17-alpha estradiol in a disperserment storage container to a required area for treatment as needed.

17. The method of claim 16, wherein the pharmaceutical composition should be applied twice per day until the desired results are obtained.
